(19) United States
(12) Patent Application Publication
(10) Pub. No.: US 2025/0317532 A1
(43) Pub. Date: Oct. 9, 2025

(54) DIGITAL HEALTH COMPANION COMMUNICATION SYSTEM WITH DYNAMIC CARE PLAN FOR USE WITH PROTECTED PERSONS

(71) Applicants: David S. Duplay, Flemington, NJ (US); Paula Muller, Keyport, NJ (US)

(72) Inventors: David S. Duplay, Flemington, NJ (US); Paula Muller, Keyport, NJ (US)

(21) Appl. No.: 18/630,019

(22) Filed: Apr. 9, 2024

(51) Int. Cl. H04N 7/14; G16H 10/20

(52) U.S. Cl. CPC H04N 7/141; G16H 10/20

(57) ABSTRACT

A digital health companion device for use by supervised or protected persons comprising a microprocessor capable of executing a protocol of instructions. The protocol of instructions may comprise sending health-related reminders to the device for the first person to hear or see; sending health and wellness education information to the device for the first person to hear or see; sending cognitive tools to the device for the first person to hear, see or interact with; or sending questions to the device regarding health and wellness for the first person to hear, see or respond to. The protocol of instructions comprises monitoring and analyzing activity on the digital health companion and either continuing or dynamically modifying the instructions going forward. The digital health companion is in electronic communication with a family caregiver app which is downloadable by a family caregiver or care provider, and can provide updated dynamic care plans and weekly, monthly or 12 month rolling digital health companion activity reports to the family caregiver app. Lastly, through the incorporation of artificial intelligence, significant events and trends will be highlighted to the family caregivers and care providers to act upon.

### FIELD OF INVENTION

The present relates to methods and devices for communication, particularly the disclosure relates to secure communication. More particularly the present invention relates to methods and devices for secure communication wherein the communication may involve one person who is under guardianship of another person. Further, the present invention relates to a device which permits constant supervision and monitoring of a person by other people or entities. The device monitors the activities and responses of the supervised person and analyzes those activities and responses to update the plan for the care and supervision of the person accordingly.

### BACKGROUND OF THE INVENTION

People are increasingly relying upon technology to stay connected with loved ones, their peers, healthcare providers, etc. With systems such as email, text messages, video conferencing, and new media becoming mainstream, formerly conventional methods of correspondence are being used less frequently. This is in part because technology allows us to connect more quickly and share more information than customary methods. For example, by using technology we can send pictures, audio files, and even video conference with people at the touch of a button. Even medical or service providers and benefits may be managed primarily online.

U.S. Pat. No. 10,848,711 to Muller discloses a method of communication between persons which comprises communication between at least two entities. The communications comprise a first person, at least one second person and at least one third person. The actual communication occurs between the first person and the third person(s). Each person has a unique identity within the system. The third person(s) may be the same person(s) as the second persons(s), the second person must know the identity of the third person and must grant approval for the communication with the first person to proceed. The communication is conducted by a first device that is coupled to a second device by electronic means; wherein the first device is in the presence of the first person and the second device is in the presence of the third person. Furthermore, the first device is always active when it is in the presence of the first person.

The communication in the '711 patent can be vocal. In one embodiment, the device is equipped with a microphone capable of transducing sound and a speaker capable of transmitting sound. The communication in the '711 patent can be visual. In one embodiment, the device is equipped with a camera capable of transducing images and a display capable of displaying transmitted images. The communication in the '711 patent can involve touch. In this embodiment, the device is equipped with touch-sensitive display capable of displaying images and transducing touch.

The first person of the '711 patent can be a child. The first person of the '711 patent can also be a person over the age of 65. The first person of the '711 patent can be a person who has been legally determined to incompetent. The first person of the '711 patent can be a person who has been medically determined to have diminished mental functions.

In another preferred embodiment of the '711 patent, the device is configured to recognize a PIN as a code by which the second person can grant approval for a third person to communicate with the first person. In a particularly preferred embodiment of the '711 patent, the third person communicates a PIN to the device to confirm the third person has granted approval for communication with the first person.

U.S. Pat. No. 10,848,711 issued on Nov. 24, 2020 to Muller is hereby incorporated by reference in its entirety.

### SUMMARY OF THE INVENTION

The present invention provides a much-needed dynamic care plan which collects data and health analytics within the digital health companion device and uses the data to modify a patient care plan in the digital health companion. The data may include a social determinants of health analysis; a population health analysis; medicare and/or medicaid care plan reporting, which may include a minimum data sheet; identification of patterns and trends at the individual level as well as the collective level within the community and cross-sectional across populations; as well as a healthcare professional patient report review for insurance billing. In one preferred embodiment, the dynamic care plan includes artificial intelligence (“AI”) enhanced data analytics and related care plan modifications.

The present invention includes providing a patient or a first person with a digital health companion and providing one or more caregivers or second and/or third persons with a companion app for use on their cellular phone or computer. The digital health companion potential customers include a full range from single community settings to multi-community settings worldwide. The invention may be used in senior living communities, assisted living facilities, memory care facilities, continuing care retirement communities (“CCRCs”) and in-home care agencies and individuals. The digital health companion provides an efficient and effective way to stay connected to clients and their families 24 hours per day and 7 days per week. The digital health companion has been designed from the ground up for non-technical aging adults. CareLink360 is one of the global leaders in providing users with Virtual Social Therapy®, important reminders, health and wellness education, and cognitive tools.

In one embodiment, the present invention comprises a device for protected communication between persons comprising at least one sensor; at least one audio speaker or video display; wherein the audio speaker is capable of transforming an electronic signal into sound; wherein the video display is capable of transforming an electronic signal into an image; at least one signal transducer; a server capable of transmitting an electronic signal from the device to an electronically coupled network; a microprocessor capable of executing a protocol of instructions and controlling the transmission of the signal through the server; wherein at least one sensor, at least one audio speaker, at least one signal transducer, at least one server and at least one microprocessor are electronically coupled together; wherein the protocol of instructions allows for protected communication between at least two entities; wherein there is a first person, there is at least one second person and there is at least one third person; wherein the protected communication is between the first person and at least one third person; wherein the first person has a unique identity; and wherein the first person's unique identity can be verified by at least one known identifiable characteristic; wherein all second persons each have a unique identity; and wherein all second persons' identities are different from the first person's identity; wherein all third persons each have a unique identity and wherein all third persons' identities are different from the first person's identity; wherein at least one second person knows the identity of a third person and has granted approval for that third person to participate in communicating with the first person prior to the initiation of communication with the first person; wherein communication is conducted by the first person to the third person through a first device that is coupled to a second device by electronic means; wherein at least one sensor is capable of identifying the unique identity of the first person by recognizing at least one physical characteristic of the first person by electronic means; wherein the first device is always active and connected electronically to a server when it is able to sense the first person by at least one sensor; wherein the protocol of instructions further comprises monitoring and analyzing activity on the device and either continuing or dynamically modifying the instructions.

In certain preferred embodiments, the protocol of instructions further comprises one or more of the following: sending health-related reminders to the device for the first person to hear or see; sending health and wellness education information to the device for the first person to hear or see; sending cognitive tools to the device for the first person to hear, see or interact with; and sending questions to the device regarding health and wellness for the first person to hear, see or respond to.

In one of the preferred embodiments, the protocol of instructions further comprises generating weekly, monthly and 12-month rolling reports of the activity on the device. In another preferred embodiment, the protocol of instructions further comprises monitoring and analyzing the activity on the device, modifying the instructions and generating a modified care plan dynamically.

In one embodiment, the present invention comprises a digital health companion device comprising: at least one sensor; at least one audio speaker or video display; wherein the audio speaker is capable of transforming an electronic signal into sound; wherein said video display is capable of transforming an electronic signal into an image; at least one signal transducer; a server capable of transmitting an electronic signal from the device to an electronically coupled network; a microprocessor capable of executing a protocol of instructions and controlling the transmission of said signal through the server; wherein at least one sensor, at least one audio speaker, at least one signal transducer, at least one server and at least one microprocessor are electronically coupled together; wherein the protocol of instructions allows for protected communication between at least two entities; wherein there is a first person and there is at least one second person; wherein the digital health companion is in electronic communication with a family caregiver companion app which is downloadable by the second person; wherein the protocol of instructions further comprises at least one of the following: sending health-related reminders to the device for the first person to hear or see; sending health and wellness education information to the device for the first person to hear or see; sending cognitive tools to the device for the first person to hear, see or interact with; or sending questions to the device regarding health and wellness for the first person to hear, see or respond to; wherein the protocol of instructions comprises monitoring and analyzing activity on the digital health companion and either continuing or modifying the instructions going forward; wherein the protocol of instructions further comprises instructing the family caregiver companion app to continue or modify the instructions going forward and the details of any modification.

In one preferred embodiment, the protocol of instructions further comprises generating weekly, monthly and 12-months rolling reports of activity on the digital health companion. In another preferred embodiment, the protocol of instructions further comprises communicating details of activity on the digital health companion to the family caregiver companion app. In another embodiment, the protocol of instructions further comprises using artificial intelligence to monitor and analyze activity on the digital health companion. In one embodiment, the protocol of instructions further comprises monitoring and analyzing the activity on the device, modifying the instructions and generating a modified care plan. In one preferred embodiment, the protocol of instructions further comprises artificial intelligence for monitoring and analyzing the activity of the device, and reporting on significant events and trends for the family caregivers and care providers to act upon.

In certain embodiments, the protocol of instructions further comprises one or more of the following: sending health-related reminders to the device for the first person to hear or see; sending health and wellness education information to the device for the first person to hear or see; sending cognitive tools to the device for the first person to hear, see or interact with; and sending questions to the device regarding health and wellness for the first person to hear, see or respond to.

In another embodiment, the present invention comprises a digital health companion device comprising: at least one sensor; at least one audio speaker or video display; wherein said audio speaker is capable of transforming an electronic signal into sound; wherein said video display is capable of transforming an electronic signal into an image; at least one signal transducer; a server capable of transmitting an electronic signal from the device to an electronically coupled network; a microprocessor capable of executing a protocol of instructions and controlling the transmission of said signal through said server; wherein at least one sensor, at least one audio speaker, at least one signal transducer, at least one server and at least one microprocessor are electronically coupled together; wherein there is a first person using the digital health companion and there is at least one second person; wherein the digital health companion is in electronic communication with a family caregiver companion app which is downloadable by the second person; wherein the protocol of instructions further comprises at least one of the following: allowing for protected communication between at least two entities; sending health-related reminders to the device for the first person to hear or see; sending health and wellness education information to the device for the first person to hear or see; sending cognitive tools to the device for the first person to hear, see or interact with; or sending questions to the device regarding health and wellness for the first person to hear, see or respond to; wherein the protocol of instructions comprises monitoring and analyzing activity on the digital health companion and either continuing or dynamically modifying the instructions to the digital health companion going forward; and wherein the protocol of instructions further comprises instructing the family caregiver companion app to continue or modify the instructions to the digital health companion going forward and the details of any modification.

In a preferred embodiment, the present invention comprises a digital health companion wherein the protocol of instructions further comprises artificial intelligence for monitoring and analyzing the activity of the device, and reporting on significant events and trends for the family caregivers and care providers to act upon. In a further embodiment, the present invention comprises a digital health companion wherein the protocol of instructions further comprises using artificial intelligence for monitoring and analyzing the activity on the device, dynamically modifying the instructions to the digital health companion, generating a modified care plan, and communicating the modified care plan to the caregiver app.

### BRIEF DESCRIPTION OF THE FIGURES

FIG. 1 identifies the digital health companion and companion app implementation footprint.

FIG. 2 is an illustration of the digital health companion device showing a typical main screen of the program as seen from the perspective of the protected person. The digital health companion is surrounded by wording describing 5 aspects of the digital health companion and images depicting those aspects.

FIG. 3 is an illustration of the ecosystem and platform for the dynamic care plan of the digital health companion.

FIG. 4 is an illustration identifying five aspects of the digital health companion and listing a number of tools provided by the digital health companion.

FIG. 5 is an illustration identifying five aspects of the digital health companion and listing a number of education content and areas provided by the digital health companion.

FIG. 6 is an illustration identifying five aspects of the digital health companion and listing a number of benefits and activities of the Virtual Social Therapy® provided by the digital health companion.

FIG. 7 is an illustration identifying five aspects of the digital health companion and listing a number of reminders provided by the digital health companion.

FIG. 8 is illustration identifying five aspects of the digital health companion and listing a number of benefits of the data analytics provided by the digital health companion.

FIG. 9 identifies and describes a three phase approach to using the digital health companion to care for aging adults.

FIG. 10 is a demographic summary identifying several facts regarding a protected person.

FIG. 11 is a chart showing a weekly summary report for the protected person's Virtual Social Therapy®, daily acknowledgements, video and music library usage and cognitive activities when using the digital health companion.

FIG. 12 is a chart showing a weekly report for the acknowledgments and answers of the protected person using the digital heath companion.

FIG. 13 is a chart showing a weekly report for the health and wellness triggered education activities of the protected person using the digital health companion.

FIG. 14 is a chart showing a weekly report for the Virtual Social Therapy® sessions of the protected person using the digital health companion.

FIG. 15 is a chart showing a weekly report for the cognitive activities of the protected person using the digital health companion.

FIG. 16 is a chart showing a weekly report for the video and music library interactions of the protected person using the digital health companion.

FIG. 17 is a chart showing a 12 month rolling summary of the acknowledgement activities of the protected person using the digital health companion.

FIG. 18 is a chart showing a 12 month rolling summary of the Virtual Social Therapy® sessions of the protected person using the digital health companion.

FIG. 19 is a bar graph showing a 12 month rolling summary of the Virtual Social Therapy® sessions of the protected person using the digital health companion.

FIG. 20 is a chart showing a 12 month rolling summary of the cognitive activities of the protected person using the digital health companion.

FIG. 21 is a bar graph showing a 12 month rolling summary of the cognitive activities of the protected person using the digital health companion.

FIG. 22 is a chart showing a 12 month rolling summary of the video and music library interactions of the protected person using the digital health companion.

FIG. 23 is a bar graph showing a 12 month rolling summary of the video and music library interactions of the protected person using the digital health companion.

FIGS. 24-26 describe an example of caring for a protected person using a digital health companion and a dynamic care plan in accordance with the present invention.

FIG. 24 describes the status and problems of the protected person before using the digital health companion and a basic care plan to start caring for the protected person.

FIG. 25 describes a daily care plan for the protected person and a description of the dynamic care plan summarizing the activities and status of the person, as well as a description of how the dynamic care plan adjusts the daily plan for the person.

FIG. 26 shows an updated daily care plan for the protected person based on the dynamic care plan of the digital health companion in accordance with the present invention.

FIG. 27 illustrates the family caregiver companion app on a cellular phone or smartphone, which allows the caregiver to receive alerts from the digital health companion and add alerts to the digital health companion.

FIG. 28 illustrates the family caregiver companion app receiving an alert from the digital health companion.

### DETAILED DESCRIPTION OF THE INVENTION

Herein is presented a method of communication between persons which comprises communication between at least two entities. The present invention includes the use of a digital health companion device capable of implementing a dynamic care plan which monitors, stores and analyzes activities on the digital health companion. In a preferred embodiment, the invention includes both a digital health companion device and a companion app accessible on a second and/or third person's cellular phone, smartphone and/or computer. The companion app allows family caregivers and any other second person and/or third person to receive alerts and data from the digital health companion, and modify the content and tools accessible to the first person on the digital health companion. The digital health companion with the dynamic care plan can monitor and analyze the first person's activities on the digital health companion, and recommend or implement modifications to the content and tools accessible on the digital health companion. The dynamic care plan may be enhanced by AI to analyze patient activity and modify the care plan dynamically, as well as reporting on significant events and trends for the family caregivers and care providers to act upon.

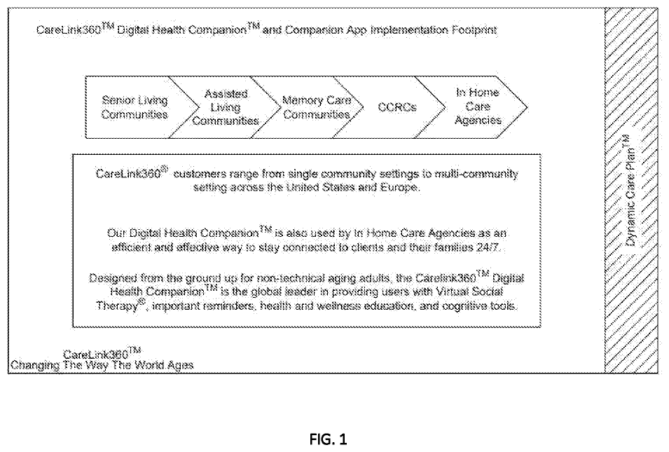

FIG. 1 describes the footprint of the CareLink360 digital health companion as including customers ranging from single community settings to multi-community settings, such as senior living communities, assisted living facilities, memory care communities, continuing care retirement communities, and in-home care agencies and individuals.

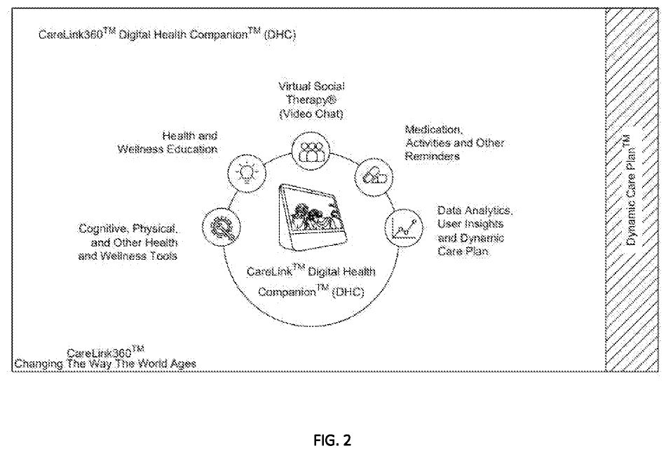

FIG. 2 illustrates five primary areas of activities which the digital health companion provides to the user. Namely, Virtual Social Therapy® (video chat); medication, activities and other reminders; health and wellness education; cognitive, physical, and other health and wellness tools; and data analytics, user insights and dynamic care plan.

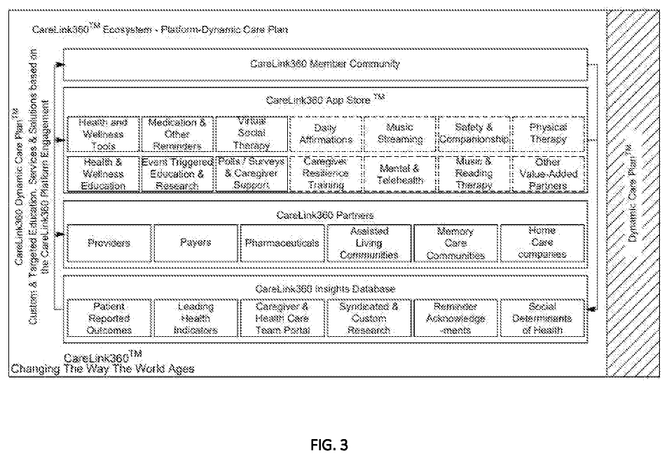

FIG. 3 describes the CareLink360 ecosystem and platform for the dynamic care plan system of the digital health companion. FIG. 3 identifies members of the CareLink360 member community, including the CareLink360 app store, the CareLink360 partners, and the CareLink360 artificial intelligence (“AI”) enhanced reporting and insights database. More particularly, FIG. 3 states that the CareLink360 app store may include choices such as health and wellness tools, medication and other reminders, Virtual Social Therapy®, health and wellness education, event triggered education and research, polls and surveys and caregiver support, daily affirmations, music streaming, safety and companionship, physical therapy, caregiver resilience training, mental and telehealth, music and reading teletherapy, and other value-added partners.

The CareLink360 partners listed on FIG. 3 are providers, payers, pharmaceuticals, assisted living communities, memory care companies and health care companies. The CareLink360 AI-enhanced reporting and insights database identified are patient reported outcomes, leading health indicators, caregiver and health care team portal, syndicated and custom research, reminder acknowledgements, and social determinants of health. Together, the CareLink360 member community, app store, partners and AI-enhanced reporting and insights database comprise the CareLink360 dynamic care plan which provides custom and targeted education, services and solutions based on the CareLink360 platform engagement.

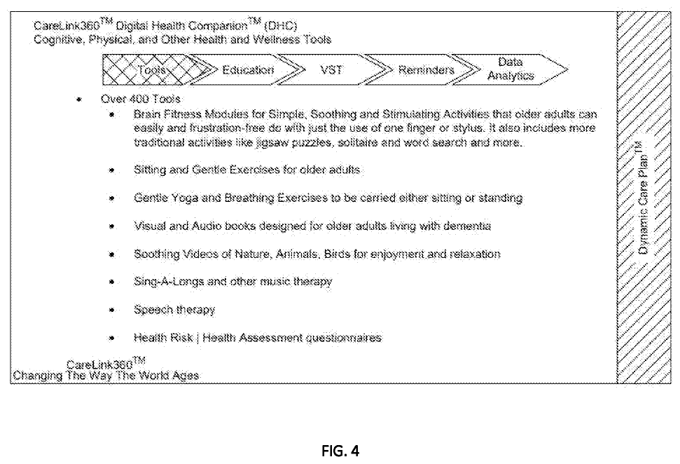

FIG. 4 describes certain details of the CareLink360 digital health companion cognitive, physical, and other health and wellness tools. FIG. 4 identifies 5 aspects of the present invention, including tools, education, Virtual Social Therapy® (“VST”, video chat), reminders and data analytics. The figure states that over 400 tools may be available, including brain fitness modules for simple, soothing and stimulating activities that older adults can perform easily and frustration-free with the use of one finger or stylus. It also includes more traditional activities such as jigsaw puzzles, solitaire, word search and more. Also listed are sitting and gentle exercises for older adults; gentle yoga and breathing exercises for either sitting or standing positions; visual and audio books designed for older adults living with dementia; soothing videos of nature, animals, birds for enjoyment and relaxation; sing-a-long songs and other music therapy; speech therapy; and health risk and health assessment questionaires.

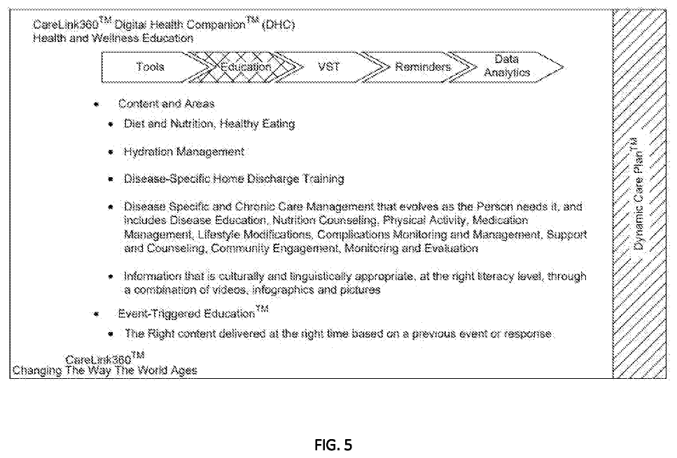

FIG. 5 describes certain details of the health and wellness education aspects of the CareLink360 digital health companion. The content and areas identified include diet and nutrition; healthy eating; hydration management; disease-specific home discharge training; disease specific and chronic care management that evolves based on the person's needs, including disease education, nutrition counseling, physical activity, medication management, lifestyle modifications, complications monitoring and management, support and counseling, community engagement, monitoring and evaluation. The figure also describes information which is culturally and linguistically appropriate, at the right literacy level, through a combination of videos, infographics and pictures. The innovative concept of Event-Triggered education is also described, which means delivering the right content at the right time based on one or more previous events and/or responses. With this, there is assurance that the content's recipient is attending and absorbing the information delivered.

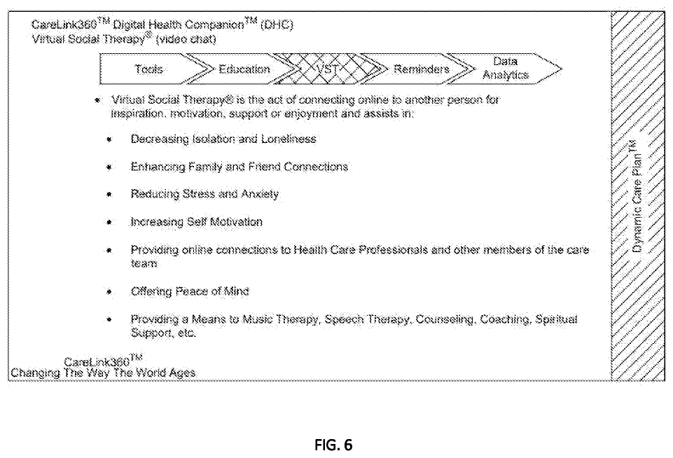

FIG. 6 describes details of the Virtual Social Therapy® (“VST”) aspects of the present invention. The figure states that Virtual Social Therapy® is the act of connecting online to another person for inspiration, motivation, support and/or enjoyment and assists in decreasing isolation and loneliness, enhancing family and friend connections, reducing stress and anxiety, increasing self-motivation, providing online connections to health care professionals and other members of the care team, offering a peace of mind, and providing access to music therapy, speech therapy, counseling, coaching, and spiritual support.

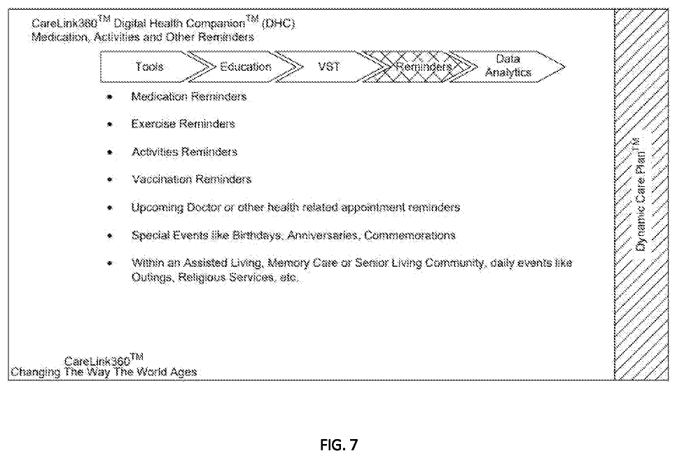

FIG. 7 describes the CareLink360 digital health companion medication, activities and other reminders, and identifies medication reminders, exercises, activities reminders, vaccination reminders, upcoming doctor and other health-related appointment reminders, special events like birthdays, anniversaries, or commemorations, and daily events such as outings or religious services within an assisted living, memory care or senior living community.

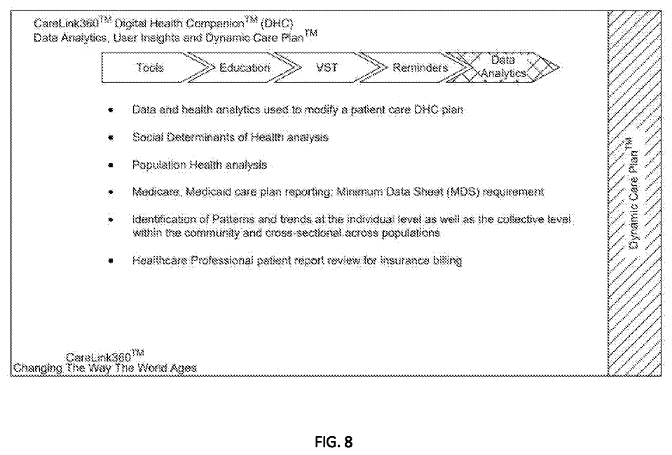

FIG. 8 describes the CareLink360 digital health companion data analytics, user insights and dynamic care plan. The data analytics identified include data and health analytics used to modify a patient care digital health companion plan dynamically; social determinants of health analysis; population health analysis; medicare or medicaid care plan reporting, including minimum data sheet requirements; identification of patterns and trends at the individual level as well as the collective level within the community and cross-sectional across populations; and health care professional patient report review for insurance billing.

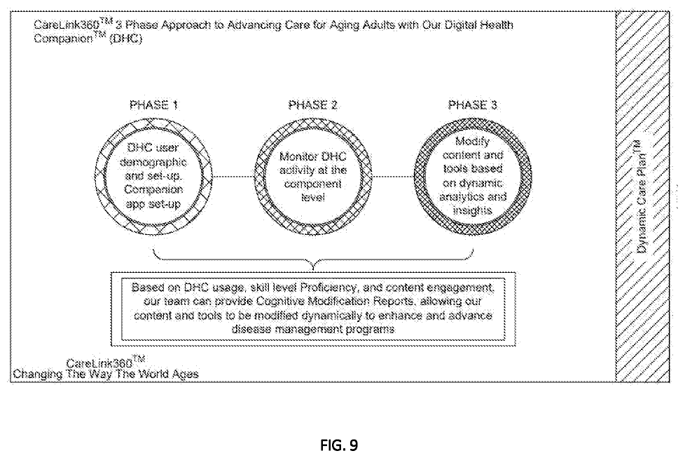

FIG. 9 describes the CareLink360 3 phase approach to advancing care for aging adults with their digital health companion. Phase 1 includes entering the first person's or patient's demographic information and setting-up the digital health companion. The companion app to be used by the second and/or third persons is also set-up. In phase 2, the activity on the digital health companion (DHC) is tracked at the component level. Phase 3 modifies the content and tools based on the dynamic analytics and insights, enhanced by artificial intelligence. Based on the usage of the DHC, the skill level proficiency and content engagement, cognitive modification reports may be provided, allowing the content and tools of the DHC to be modified dynamically to enhance and advance disease management programs.

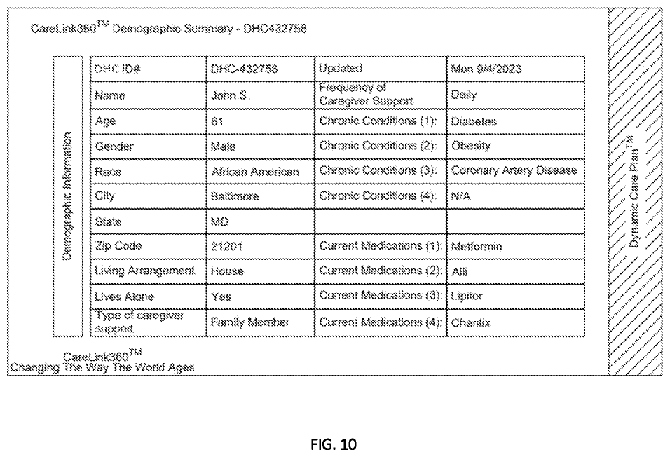

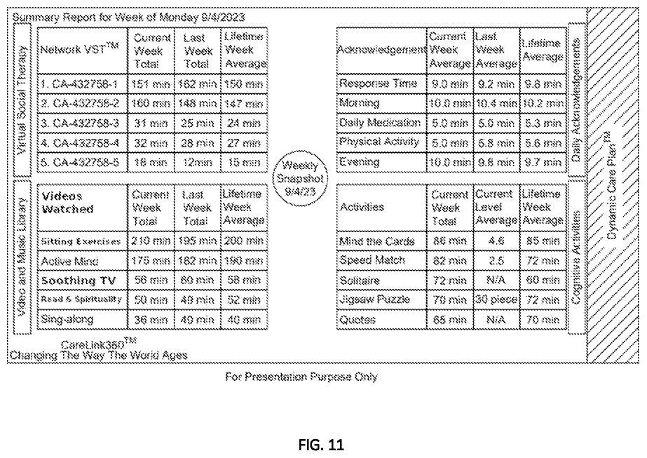

FIG. 10 sets forth the demographic data for a DHC user named John S., including frequency of caregiver support, several chronic conditions, several medications, and living arrangements. FIG. 11 sets forth a weekly summary report for the same DHC user John S., including the time spent on Virtual Social Therapy®, the time spent using the video and/or music library of the DHC, the time taken to acknowledge and act on daily reminders such as daily medication or physical activities reminders, and the time spent and level of cognitive activities such as solitaire and/or jigsaw puzzles.

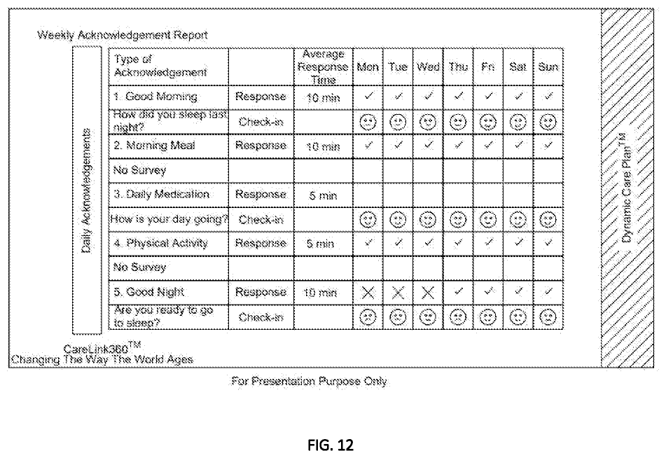

FIG. 12 is a weekly report of the DHC user's daily acknowledgements, including acknowledging having a morning meal, taking daily medication, performing physical activity, and checking-in regarding how they slept last night, how their day is going and readiness to go to sleep.

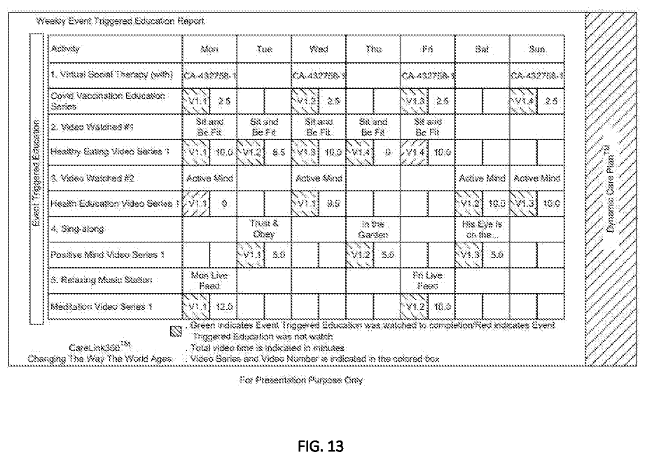

FIG. 13 is a chart setting forth a weekly report for event triggered education, including Virtual Social Therapy®, watching a covid vaccination education series episode on the DHC, watching videos regarding healthy eating, watching a sing-along positive mind video, and listening to a relaxing music station and watching a meditation video.

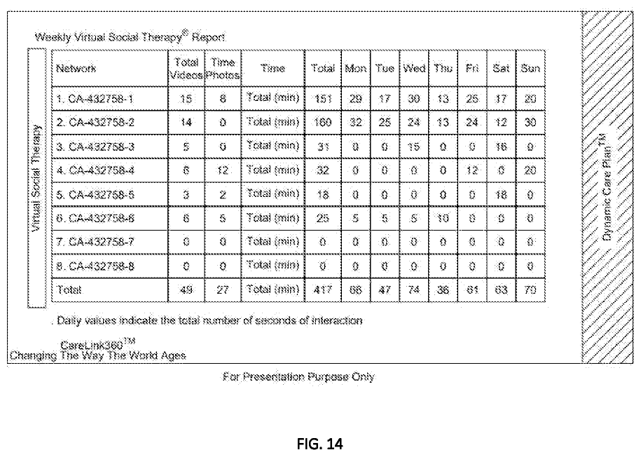

FIG. 14 is a weekly Virtual Social Therapy® report showing that there were 8 family members and/or care providers in the network available on the DHC, the total number of videos and pictures sent by each family member or care provider in the network, as well as the amount of time spent on the Virtual Social Therapy® sessions per day and the total for the week for each member of the family and care provider network.

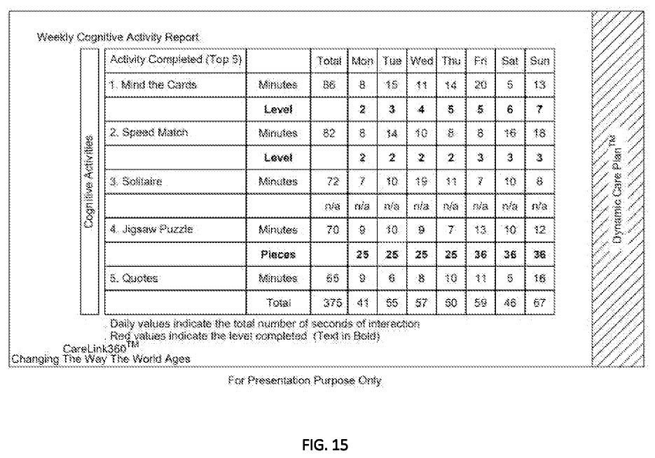

FIG. 15 is a weekly cognitive activity report showing the top 5 cognitive activities on the DHC, i.e. mind the cards, speed match, solitaire, jigsaw puzzle and quotes. The figure also shows the time spent on each activity every day and the difficulty level of the activity, including the total number of minutes of interaction for the week.

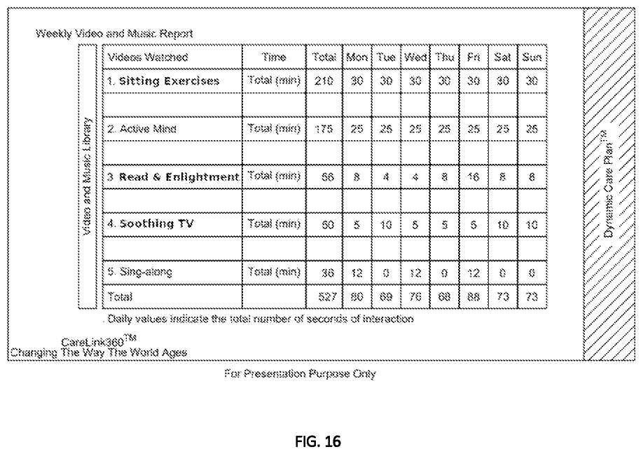

FIG. 16 is a weekly video and music library report showing 5 videos and music library selections which were watched and/or listened to by the user of the DHC, as well as the total minutes of interaction for the week.

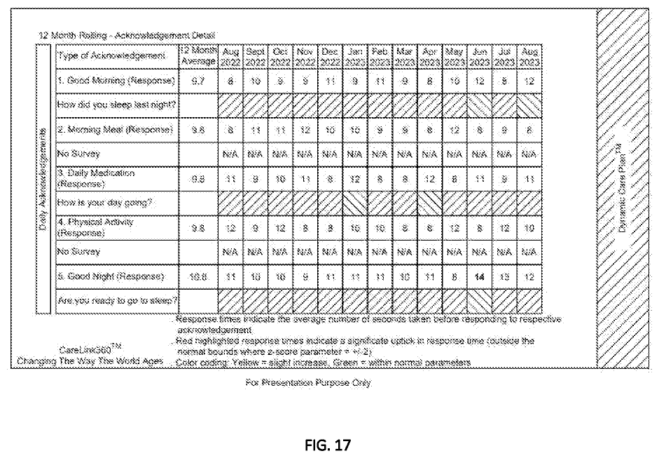

FIG. 17 is a 12 month rolling report of daily acknowledgements by the user of the DHC. The report includes good morning response, a response saying how well the person slept the prior night, acknowledgement of a morning meal, acknowledgement of taking daily medication, a response saying how the person's day is going, an acknowledgement of physical activity. A response to a good night wish, and an assessment of whether the person was ready to go to sleep.

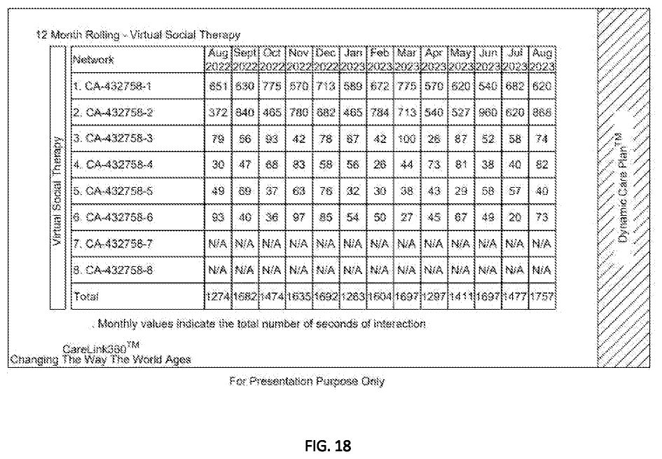

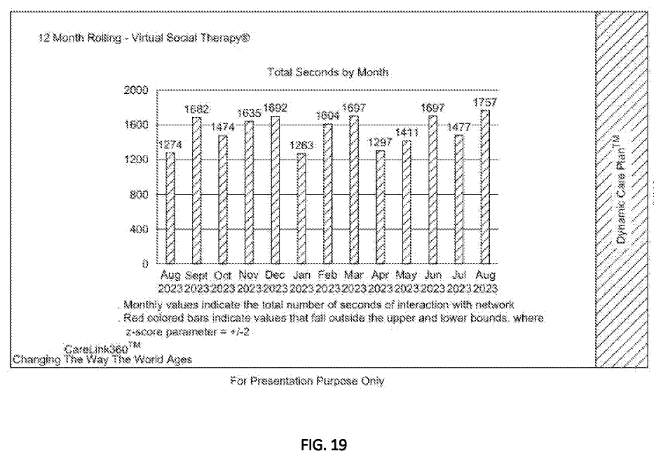

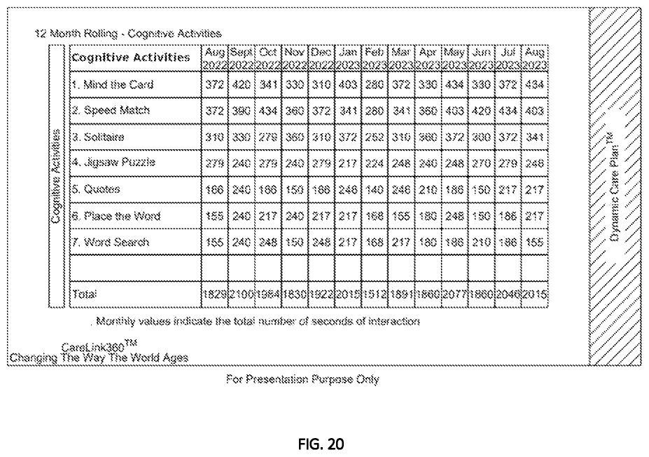

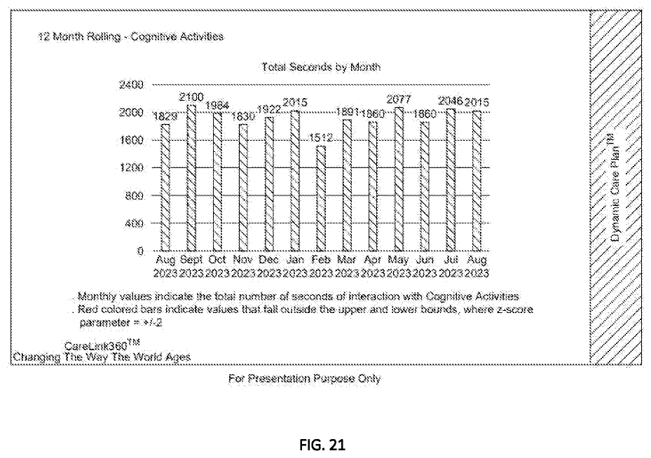

FIG. 18 is a 12 month rolling chart showing the DHC user's Virtual Social Therapy® sessions with 8 members of the family and care provider network on the DHC, including total seconds of interaction per month. Similarly, FIG. 19 is a 12 month rolling report of the DHC user's Virtual Social Therapy® interactions with each member of the network on the DHC per month. As shown in FIG. 20, the dynamic care plan of the present invention also records and analyzes the 12 month rolling report of cognitive activities for the user of the DHC, including the following activities on the DHC: mind the card, speed match, solitaire, jigsaw puzzle, quotes, place the word and word search. The total number of seconds of interaction are shown for each month for each activity. FIG. 21 is also a 12 month rolling report of cognitive activities generated by the AI-enhanced analytics of the dynamic care plan. This figure is a bar graph of the total seconds per month the DHC user has interacted with the cognitive activities.

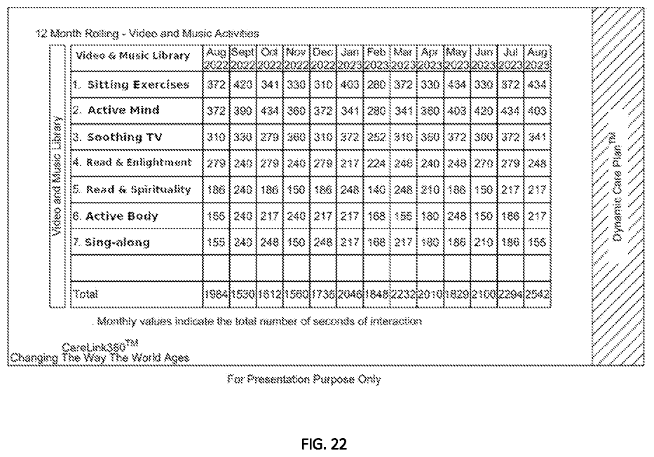

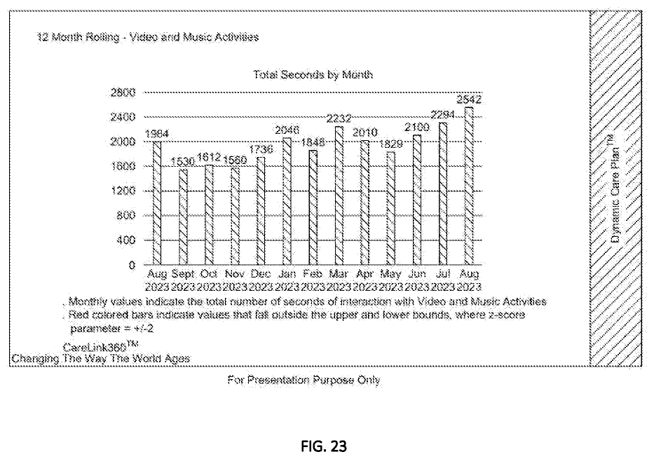

FIG. 22 is a chart detailing the 12 month rolling report for the video and music activities by the DHC user, showing total monthly seconds of interaction. Similarly, FIG. 23 is a bar graph depicting the number of seconds of interaction in video and music activities. The AI-enhanced data analytics of the DHC can process all such data and, combined with user insights and the dynamic care plan software, produce a modified updated care plan for the person.

### Dedicated Device

The dedicated device, pervasive presence, and its adaptive features and functions become a safe and secure emotional companion device for the protected person, with oversight by the second person/administrator. The protected person can rely on an ongoing and continued dialogue with the dedicated device in an easy and automatic way, because the device does not go to sleep. The dedicated device presents a view on screen or by alternative means and methods of the state of the protected person, and options that are available to the protected person, for example, an adapted interface might provide pictures of the loved ones for the protected person to touch to initiate communications, through video visits, or by sending a message, for example, provides a safe gateway to other features and services, and for other purposes.

The dedicated device presents the emotional and health state of the protected person, and solicits engagement by the protected person, as a companion would, provides helpful information, simplified access to communication, reminders, schedules, healthcare monitoring and management, and is updated automatically without intervention by the protected person. The dedicated device is a method to manage communication, monitor, articulate, and verify the protected person's state, and adapt or promote actions to improve emotional valence and social interaction, to reduce isolation, stress, and loneliness, to reduce complexity, and for the best health outcomes.

### Communication

The devices and methods described herein assist and/or allow for communication between two or more people, wherein one person is a protected person. Traditionally communication is considered to be voice communication, often supported by video. However, it is understood that communication can occur in many forms. It is understood that communication can occur with voice alone, or by text, with alternative media, a robot or similar device, with the assistance of sensors, actuators, or through specialized communications techniques for example, symbols, Braille or similar tactile means, specialized methods including speech to text to graphically generated sign language, through a relay operator or person acting in that capacity, captioning, or sign language on a video display.

It is also understood that a Telecommunication Device for the Deaf (TDD) system can be used; TDD systems are also known as teletypewriter, TTY, textphone, and minicom, MCM, Def-tone, DTS, each of these terms are used interchangeably herein.

Communication can also be through touch. This is particularly useful for some protected people for whom traditional communication by voice or text is intimidating. In one configuration software allows a person to select an image using a touch screen menu to convey thoughts or emotions. The image may be directly communicated to the recipient it may also be converted to another form of communication including voice or text. For instance, a protected person might hear their daughter's voice and select a smiley face image, or another emotional image or media to convey a sentiment or message, on the touch screen. Upon that the selection of that image, the device can be configured to say to the daughter, “I am safe and happy.” In some embodiments this voice is a computer-generated voice. In other embodiments, the voice is prerecorded by the protected person. In still other embodiments, the device is configured to synthesize a voice to sound like the protected person having derived the protected person's voice from collected recordings.

### Electronic Means or Communication

The terms “electronic means” or “electronic communication” include end to end wired, wireless, radio transmission, and others, inclusive of campus environments such as a closed system (i.e. hospitals), including specialized means such as inductive coupling (e.g, methods of data transfer between implanted medical devices and external systems), near field communications in devices of close proximity (NFC), optical, infrared, scanners, acoustic transmission of data gathered by microphones, and many other means and methods in use to enable connectivity for devices and services. The field of networking is experiencing tremendous growth and diversification, with useful implementations for all aspects of connectivity including traditional wired and wireless communications infrastructure, with additions for simple and secure networking as well as self-configuring ad hoc networking. Over time, some are more abundant and preferred in a given environment such as a medical environment, while others are prevalent because of simplicity, cost, size, safety, embedded infrastructure, practical nature of the method of transmission, etc. Some come with regulatory issues (such as medical devices), others are compliant with standards including Wi-Fi and Bluetooth, and others. Some are more ideally integrated into solutions because of the requirements of the application associated with the power supply, environmental interference issues, and more. The present invention advantages not only the existing internet worldwide communication infrastructure, wired and wireless, as well as campus systems typically found at hospitals, schools, in homes, and elsewhere, but also includes device to device communication, and newly emerging techniques and enablements as needed.

### Supervised or Protected Person

The devices and methods described herein assist and/or allow for communication between two or more people, wherein one person is a supervised or protected person. By supervised or protected person is it meant a person who is being cared for by another person, wherein the other person is actively making important decisions for the protected person. For the purpose of this disclosure, the protected person is relying on another person to make decisions, at a minimum, about the other people the protected person communicates with and when that communication is allowed to be conducted.

In some situations, the protected person is of diminished capacity. In some situations, a physician, hospital or clinic has determined that the person lacks capacity to make decisions. In some situations, a court has determined that the person's mental functions are diminished or impaired. In some situations, the court determination is statutory; for instance, a parent or guardian has legal control for their child or ward, at least until the protected person is of legal age. In many situations, the protected person has knowingly volunteered to defer control of their communication to another person for any one of a variety of personal reasons. The status of the protected person may be either permanent or temporary.

### Example 1

The present invention is further defined in the following Examples. It should be understood that these Examples while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

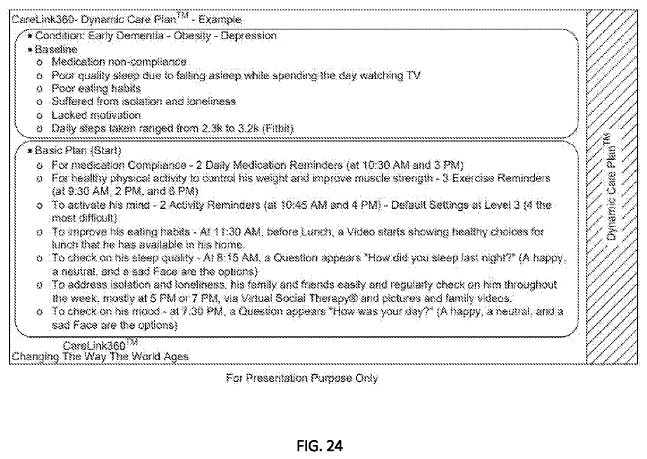

As described in FIG. 24, a man having early dementia, obesity and depression began using the CareLink360 digital health companion with dynamic care plan. His baseline conditions included medication non-compliance, poor quality sleep due to falling asleep while spending the day watching television, poor eating habits, suffered from isolation and loneliness, lacked motivation, and his daily steps taken ranged from 2.3k to 3.2k as measured by a wrist worn device. The basic plan programmed into the DHC to begin was as follows: for medication compliance there were 2 daily medication reminders at 10:30 AM and 3:00 PM; for healthy physical activity to control weight and improve muscle strength he received 3 exercise reminders at 9:30 AM, 2 PM and 6 PM; to activate his mind he receives 2 activity reminders at 10:45 AM and 4 PM with the difficulty level set at a default level 3 of 4; to improve his eating habits, at 11:30 AM before lunch a video starts showing healthy choices for lunch which are available in the person's home; in order to check on his sleep quality, at 8:15 AM a question appears on the DHC asking how he slept the night before and offering a happy, a neutral and a sad face to chose from to respond; to address isolation and loneliness his family and friends easily and regularly check on him throughout the week, mostly at 5 PM or 7 PM via Virtual Social Therapy®, pictures and family videos; to check on his mood, at 7:30 PM a question appears asking how his day was and the response options are a happy face, a neutral face or a sad face.

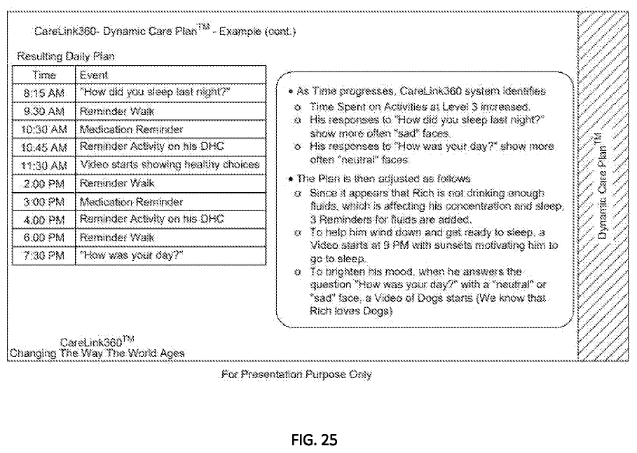

FIG. 25 includes a chart listing each of the reminders, questions and activities which the DHC presents to the person on a daily basis. The figure also states that, as time progressed, the CareLink360 DHC with dynamic care plan identified that time spent on activities at level 3 difficulty increased, the person's responses regarding how he slept the prior night were more often sad faces, and his responses regarding how his day was were more often neutral faces. The dynamic care plan was then adjusted as follows: 3 reminders for fluids were added because it appeared that the person was not drinking enough fluids, which affected his concentration and sleep; a video started playing every day at 9 PM with sunsets to help him wind down and motivate him to sleep; and to brighten his mood when he answers how his day was with a neutral face or a sad face, a video of dogs starts on the DHC (he is known to love dogs).

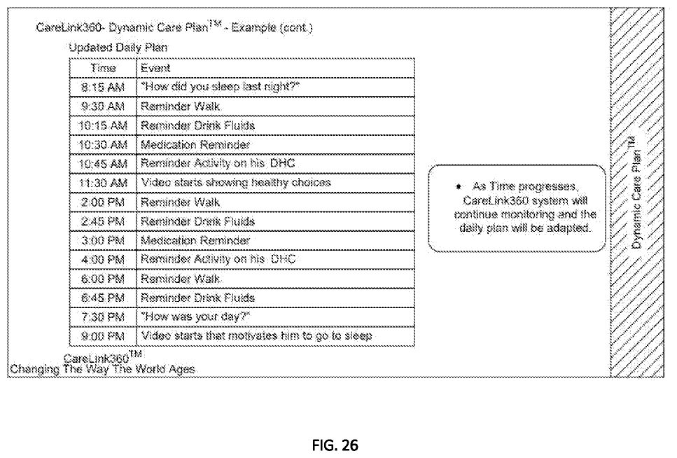

FIG. 26 sets forth a chart detailing the updated daily dynamic care plan with the reminders to drink fluids and the video to motivate sleep. The figure also states that as time progresses, the CareLink360 digital health companion with dynamic care plan will continue to monitor the user's activities and responses and the daily plan will be adapted accordingly.

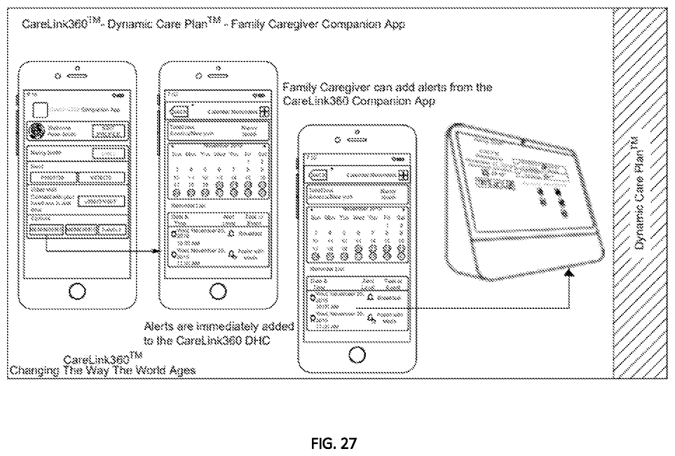

FIG. 27 illustrates the use of the family caregiver companion app wherein the family caregiver adds alerts from the CareLink360 companion app to the digital health companion.

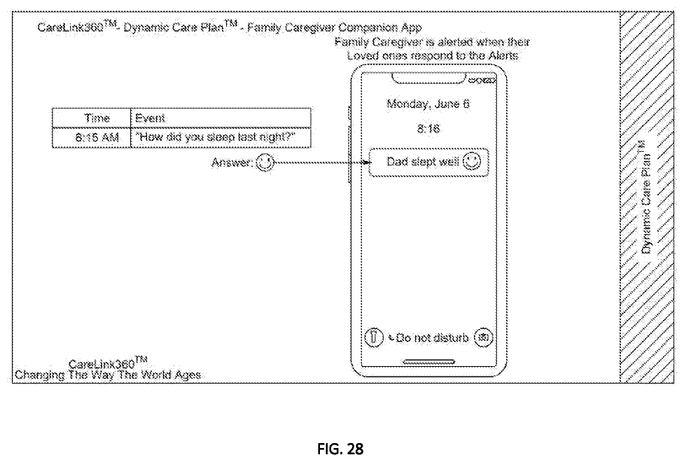

FIG. 28 shows that the family caregiver is alerted on their cell phone via the family caregiver companion app when the user responds regarding how he slept the prior night.

All illustrations of the drawings are for the purpose of describing specific embodiments of the invention and are not intended to limit the scope of the invention. The scope of the invention, therefore, is not intended to be limited to the exemplary embodiments shown and described herein. Instead, the scope and fair meaning of the invention is embodied by the claims.

We claim:

1. A device for protected communication between persons comprising:
at least one sensor;
at least one audio speaker or video display;
wherein said audio speaker is capable of transforming an electronic signal into sound;
wherein said video display is capable of transforming an electronic signal into an image;
at least one signal transducer;
a server capable of transmitting an electronic signal from the device to an electronically coupled network;
a microprocessor capable of executing a protocol of instructions and controlling the transmission of said signal through said server;
wherein at least one sensor, at least one audio speaker, at least one signal transducer, at least one server and at least one microprocessor are electronically coupled together;
wherein the protocol of instructions allows for protected communication between at least two entities;
wherein there is a first person, there is at least one second person and there is at least one third person;
wherein the protected communication is between the first person and at least one third person;
wherein the first person has a unique identity;
wherein the first person's unique identity can be verified by at least one known identifiable characteristic;
wherein all second persons each have a unique identity; and wherein all second persons' identities are different from the first person's identity;
wherein all third persons each have a unique identity and wherein all third persons' identities are different from the first person's identity;
wherein at least one second person knows the identity of a third person and has granted approval for that third person to participate in communicating with the first person prior to the initiation of communication with the first person;
wherein communication is conducted by the first person to the third person through a first device that is coupled to a second device by electronic means;
Wherein at least one sensor is capable of identifying the unique identity of the first person by recognizing at least one physical characteristic of the first person by electronic means;
wherein the first device is always active and connected electronically to a server when it is able to sense the first person by at least one sensor; and
wherein the protocol of instructions further comprises monitoring and analyzing activity on the device and either continuing or modifying the instructions.

2. The device of claim 1, wherein the protocol of instructions further comprises sending health-related reminders to the device for the first person to hear or see.

3. The device of claim 1, wherein the protocol of instructions further comprises sending health and wellness education information to the device for the first person to hear or see.

4. The device of claim 1, wherein the protocol of instructions further comprises sending cognitive tools to the device for the first person to hear, see or interact with.

5. The device of claim 1, wherein the protocol of instructions further comprises sending questions to the device regarding health and wellness for the first person to hear, see or respond to.

6. The device of claim 1, wherein the protocol of instructions further comprises generating weekly, monthly and 12 month rolling reports of all the interactions with the system and utilization of the devices and companion apps.

7. The device of claim 1, wherein the protocol of instructions further comprises monitoring and analyzing the activity on the device, dynamically modifying the instructions and generating a person-centric modified care plan.

8. A digital health companion device comprising:
at least one sensor;
at least one audio speaker or video display;
wherein said audio speaker is capable of transforming an electronic signal into sound;
wherein said video display is capable of transforming an electronic signal into an image;
at least one signal transducer;
a server capable of transmitting an electronic signal from the device to an electronically coupled network;
a microprocessor capable of executing a protocol of instructions and controlling the transmission of said signal through said server;
wherein at least one sensor, at least one audio speaker, at least one signal transducer, at least one server and at least one microprocessor are electronically coupled together;
wherein the protocol of instructions allows for protected communication between at least two entities;
wherein there is a first person and there is at least one second person;
wherein the digital health companion is in electronic communication with a family caregiver companion app which is downloadable by the second person;
wherein the protocol of instructions further comprises at least one of the following: sending health-related reminders to the device for the first person to hear or see; sending health and wellness education information to the device for the first person to hear or see; sending cognitive tools to the device for the first person to hear, see or interact with; or sending questions to the device regarding health and wellness for the first person to hear, see or respond to;
wherein the protocol of instructions comprises monitoring and analyzing activity on the digital health companion and either continuing or dynamically modifying the instructions going forward; and
wherein the protocol of instructions further comprises instructing the family caregiver companion app to continue or modify the instructions going forward and the details of any modification.

9. The digital health companion of claim 8, wherein the protocol of instructions further comprises generating weekly, monthly and 12 month rolling reports of all interactions with the system and utilization of the digital health companion and companion apps.

10. The digital health companion of claim 8, wherein the protocol of instructions further comprises communicating details of activity on the digital health companion to the family caregiver companion app.

11. The digital health companion of claim 8, wherein the protocol of instructions further comprises using artificial intelligence to monitor and analyze activity on the digital health companion.

12. The digital health companion of claim 8, wherein the protocol of instructions further comprises sending health-related reminders to the device for the first person to hear or see.

13. The digital health companion of claim 8, wherein the protocol of instructions further comprises sending health and wellness education information to the device for the first person to hear or see.

14. The digital health companion of claim 8, wherein the protocol of instructions further comprises sending cognitive tools to the device for the first person to hear, see or interact with.

15. The digital health companion of claim 8, wherein the protocol of instructions further comprises sending questions to the device regarding health and wellness for the first person to hear, see or respond to.

16. The digital health companion of claim 8, wherein the protocol of instructions further comprises monitoring and analyzing the activity on the device, modifying the instructions and generating a modified care plan dynamically.

17. The digital health companion of claim 16, wherein the protocol of instructions further comprises artificial intelligence for monitoring and analyzing the activity on the device, assisting in generating a modified person centric care plan, and reporting on significant events and trends for the family caregivers and care providers to act upon.

18. A digital health companion device comprising:
at least one sensor;
at least one audio speaker or video display;
wherein said audio speaker is capable of transforming an electronic signal into sound;
wherein said video display is capable of transforming an electronic signal into an image;
at least one signal transducer;
a server capable of transmitting an electronic signal from the device to an electronically coupled network;
a microprocessor capable of executing a protocol of instructions and controlling the transmission of said signal through said server;
wherein at least one sensor, at least one audio speaker, at least one signal transducer, at least one server and at least one microprocessor are electronically coupled together;
Wherein there is a first person using the digital health companion and there is at least one second person;
wherein the digital health companion is in electronic communication with a family caregiver companion app which is downloadable by the second person;
wherein the protocol of instructions further comprises at least one of the following: allowing for protected communication between at least two entities; sending health-related reminders to the device for the first person to hear or see; sending health and wellness education information to the device for the first person to hear or see; sending cognitive tools to the device for the first person to hear, see or interact with; or sending questions to the device regarding health and wellness for the first person to hear, see or respond to;
wherein the protocol of instructions comprises monitoring and analyzing activity on the digital health companion and either continuing or dynamically modifying the instructions to the digital health monitor going forward; and
wherein the protocol of instructions further comprises instructing the family caregiver companion app to continue or modify the instructions to the digital health companion going forward and the details of any modification.

19. The digital health companion of claim 18, wherein the protocol of instructions further comprises artificial intelligence for monitoring and analyzing the activity of the device.

20. The digital health companion of claim 19, wherein the protocol of instructions further comprises monitoring and analyzing the activity on the device, modifying the instructions to the digital health companion, generating a modified care plan, and communicating the modified care plan to the caregiver app.
